(19) United States
(12) Patent Application Publication
(10) Pub. No.: US 2010/0240760 A1
(43) Pub. Date: Sep. 23, 2010

(54) Biomarkers for Efficacy of Aliskiren as a Hypertensive Agent

(76) Inventors: Jessie Gu, Framingham, MA (US); Joanne Meyer, Framingham, MA (US)

Correspondence Address: DILWORTH & BARRESE, LLP, 1000 WOODBURY ROAD, SUITE 405, WOODBURY, NY, 11797, US

(21) Appl. No.: 11/909,202

(22) PCT Filed: Mar. 20, 2006
(86) PCT No.: PCT/US06/09913

§ 371 Date: Sep. 20, 2007

Related U.S. Application Data
(60) Provisional application No. 60/664,248, filed on Mar. 22, 2005.
(60) Provisional application No. 60/742,401, filed on Feb. 6, 2006.

(51) Int. Cl. A61K 31/165; C12Q 1/68; A61P 9/12

(52) U.S. Cl. 514/616; 435/6

(57) ABSTRACT

A retrospective pharmacogenetic analysis was conducted in an attempt to evaluate potential association between genetic variation and outcome of a clinical trial of efficacy of aliskiren as an antihypertensive agent. Forty-eight polymorphisms were examined in twelve genes from the renin-angiotensin-aldosterone system (RAS) or previously implicated in blood pressure regulation. Significant associations were seen between one polymorphism in the angiotensin converting enzyme (ACE) gene, two polymorphisms in the angiotensin II type 2 receptor (AGTR2) gene, and clinical parameters of mean sitting diastolic and systolic blood pressure decrease. These effects were not found with irbesartan and placebo treatment, but instead were specific to aliskiren treatment.

### FIELD OF THE INVENTION

This invention relates generally to the analytical testing of tissue samples in vitro, and more particularly to aspects of genetic polymorphisms indicative of the efficacy of aliskiren as an anti-hypertensive agent.

### BACKGROUND OF THE INVENTION

The renin angiotensin system (RAS) plays an important role in the regulation of blood pressure and volume homeostasis. Renin is secreted by the kidney in response to a decrease in circulating volume and blood pressure, and cleaves the substrate angiotensinogen to form the inactive decapeptide angiotensin I (Ang I). Ang I is converted to the active octapeptide Ang II by angiotensin converting enzyme (ACE). Ang II interacts with cellular receptors inducing vasoconstriction and release of catecholamines from the adrenal medulla and pre-junctional nerve endings. It also promotes aldosterone secretion and sodium reabsorption. In addition, Ang II inhibits renin release, thus providing a negative feedback to the system. Accordingly, Ang II acts at various levels (e.g. vasculature, sympathetic nervous system, cortex and medulla of the adrenal gland) to increase vascular resistance and blood pressure.

The renin angiotensin system (RAS) can be blocked at various levels. Since renin inhibitors block the RAS at a higher level than ACE inhibitors and Ang II antagonists, they have a different effect on the components of the RAS. After the administration of a renin inhibitor, the formation of both Ang I and Ang II is blocked. Whereas after ACE inhibition, only Ang II formation is blocked and levels of Ang I increase. Ang I is thus still available to be converted to Ang II and other angiotensin peptides by other pathways such as the chymase system.

Aliskiren (SPP100) is a non-peptide antihypertensive agent with a low molecular weight (609.8). See, Wood J M et al., Biochem. Biophys. Res. Commun. 308(4):698-705 (Sep. 5, 2003). The mechanism of action is different from other antihypertensive on the market. Aliskiren blocks the renin angiotensin system (RAS) at its first and rate-limiting step. In vitro, aliskiren is a potent inhibitor of human renin (IC₅₀=0.6 nM). In vivo, aliskiren administered both orally (p.o.) or intravenously (i.v.) in several studies with sodium-depleted marmosets caused complete inhibition of plasma renin activity (PRA), sustained reductions in mean arterial pressure (MAP), and significant increases in plasma concentrations of active and total renin. In humans, plasma concentrations of aliskiren increase rapidly after administration reaching peak levels in 3-5 hours. Both Cₘₐₓ and AUC increase with dose, but in a non-linear fashion. The half-life of aliskiren is approximately 25 hours and its bioavailability is approximately 2.7%.

Conventional medical approaches to diagnosis and treatment of disease are based on clinical data alone or made in conjunction with a diagnostic test. Such traditional practices often lead to therapeutic choices that are not optimal for the efficacy of the prescribed drug therapy or to minimize the likelihood of side effects for an individual subject. Therapy specific diagnostics (a.k.a., theranostics) is an emerging medical technology field, which provides tests useful to diagnose a disease, choose the correct treatment regime and monitor a subject's response. That is, theranostics are useful to predict and assess drug response in individual subjects, i.e., individualized medicine. Theranostic tests are also useful to select subjects for treatments that are particularly likely to benefit from the treatment or to provide an early and objective indication of treatment efficacy in individual subjects, so that the treatment can be altered with a minimum of delay.

Progress in pharmacogenetics, which establishes correlations between responses to specific drugs and the genetic profile of individual patients, is foundational to the development of new theranostic approaches. As such, there is a need in the art for the evaluation of patient-to-patient variations in gene sequence and gene expression. A common form of genetic profiling relies on the identification of DNA sequence variations called single nucleotide polymorphisms (“SNPs”), which are one type of genetic mutation leading to patient-to-patient variation in individual drug response. It follows that there is a need in the art to identify and characterize genetic mutations, such as SNPs, which are useful to identify the genotypes of subjects associated with drug responsiveness.

### SUMMARY OF THE INVENTION

The present invention provides a response to the need in the art. Significant associations are identified between polymorphisms in the angiotensin converting enzyme (ACE) gene, polymorphisms in the angiotensin II type 2 receptor (AGTR2) gene, and clinical parameters of mean sitting diastolic and systolic blood pressure decrease following treatment with aliskiren as an antihypertensive agent. These effects are not found with irbesartan and placebo treatment, but are instead specific to aliskiren treatment.

Accordingly, the invention provides for the use of aliskiren in the manufacture of a medicament for the treatment of hypertension in a selected patient population. The patient population to be treated is selected on the basis of genetic polymorphisms in biomarker genes present in the patients. The biomarker genes are the angiotensin converting enzyme (ACE) gene and the angiotensin II type 2 receptor (AGTR2) gene. The genetic polymorphisms are indicative of the efficacy of aliskiren in treating hypertension.

The invention also provides a diagnostic method for determining the responsiveness of an individual with hypertension to treatment with aliskiren, based upon the identity of a nucleotide pair at one or more of the polymorphic genetic loci of the invention.

The invention further provides a theranostic method for treating hypertension in an individual. An antihypertensive agent is administered to the individual if the nucleotide pair at the polymorphic genetic loci of the invention indicate that the individual is responsive to treatment with an antihypertensive agent. In one embodiment, the antihypertensive agent is aliskiren. An alternative therapy is administered to the individual if the nucleotide pair at the polymorphic genetic loci of the invention indicate that the individual is not responsive to treatment with an antihypertensive agent.

The invention generally provides a method for reducing daytime ambulatory diastolic blood pressure (DADBP). In a specific embodiment, the invention provides a theranostic method of reducing mean sitting diastolic blood pressure (MSDBP).

In addition, the invention provides a method for reducing daytime ambulatory systolic blood pressure (DASBP). In as specific embodiment, the invention provides a theranostic method of reducing mean sitting systolic blood pressure (MSSBP).

Moreover, the invention provides a method for choosing individuals for inclusion in a clinical trial for determining the efficacy of an antihypertensive agent for the treatment of hypertension. An individual can be included in the trial if the genotype of the individual is indicative of efficacy of the antihypertensive agent for treating hypertension in that individual. The individual can be withdrawn from inclusion in the trial if the genotype of the individual is not indicative of efficacy of the antihypertensive agent for treating hypertension in that individual.

The invention provides kits for the practice of the methods of the invention. The invention also provides a way to use the angiotensin converting enzyme (ACE) gene product and the angiotensin II type 2 receptor (AGTR2) gene product as targets for drug discovery.

### BRIEF DESCRIPTION OF THE DRAWINGS

FIG. 1 is a set of bar graphs showing the responder ratio segregated by SNP_{—}4769 genotypes for the three aliskiren treated groups together, the highest dose aliskiren group (600 mg), the irbesartan group and the placebo group. In the rows at the bottom of the figure, the top row refers to the CT allele and the bottom row refers to the TT allele.

### DETAILED DESCRIPTION OF THE INVENTION

A retrospective pharmacogenetic analysis was conducted in an attempt to evaluate potential association between genetic variation and clinical outcome in a clinical trial. In the clinical trial, aliskiren at 75, 150, or 300 mg administered once daily was shown to be an effective antihypertensive agent in mild-to-moderate hypertensive patients resulting in a statistically significant reduction in daytime ambulatory systolic blood pressure (DASBP). All doses of active treatment were statistically superior to placebo in reducing mean sitting diastolic blood pressure (MSDBP) at the clinical trial endpoint and in the intend-to treat (ITT) population at week 8, and in the per protocol population at clinical trial endpoint. Similar MSDBP reductions were achieved with aliskiren 150 mg and irbesartan 150 mg. See, EXAMPLE I, below.

For mean sitting systolic blood pressure (MSSBP), all doses of active treatment were statistically superior to placebo at the clinical trial endpoint. Aliskiren 300 and 600 mg were statistically superior to placebo and irbesartan at the clinical trial endpoint. Similar MSSBP reductions were achieved with aliskiren 150 mg and irbesartan 150 mg. Aliskiren 300 and 600 mg produced the greatest reductions. See, EXAMPLE I, below.

In the pharmacogenetic analysis, forty-eight polymorphisms were examined in twelve genes from the renin-angiotensin-aldosterone system (RAS) or previously implicated in blood pressure regulation. Significant associations were seen between one polymorphism in the angiotensin converting enzyme (ACE) gene (SNP_{—}4769, SEQ ID NO:1), two polymorphisms in the angiotensin II type 2 receptor (AGTR2) gene (SNP_{—}1445, SEQ ID NO:2 and SNP_{—}4795, SEQ ID NO:3), and clinical parameters of mean sitting diastolic and systolic blood pressure decrease. These effects were not found with irbesartan and placebo treatment, rather they were specific to aliskiren treatment in this analysis.

The nucleotide sequence of SNP_{—}4769 (SEQ ID NO:1) is as follows:

AGGACTTCCC AGCCTCCTCT TCCTGCTGCT CTGCTACGGG
CACCCTCTGC TGGTCCCCAG CCAGGAGGCA/Y CCCAACAGGT
GACAGTCACC CATGGGACAA GCAGCCAGGC AACAACCAGC
AGCCAGACAA CCACCCACCA

SNP_{—}4769 is a coding SNP which changes the amino acid sequence from proline to serine at codon 32 in the ACE enzyme.

The nucleotide sequence of SNP_{—}1445 (SEQ ID NO:2) is as follows:

TGGAAACTTC ATTTTTTTTG TTTGAGATTT ATTTGAATGA
GCTGTTATGA TTGGAGACAG TGAGAATTTC AGATTAATGT
TTTGCAGACA AAAAAAAACC TCTCTGGAAA GCTGGCAAGG
GTTCATAAGT CAGCCCTAGA ATTATGTAGG TTGAAGGCTC
CCAGTGGACA GACCAAACAT ATAAGAAGGA AACCAGAGAT
CTGGTGCTAT TACGTCCCAG CGTCTGAGAG AACGAGTAAG
CACAGAATTC AAAGCATTCT GCAGCCTGAA TTTTGAAGGT
AAGTATGAAC AATTTATATA TAATTTACTT GGAAAGTAGA
ACATACATTA AATGAAAATA TTTTTTATGG ATGAACTTCT
GTTTTTCCTG TGTTTTAACA CTGTATTTTG CAAAACTCCT/R
AATTATTTAG CTGCTGTTTC TCTTACAGGA GTGTGTTTAG
GCACTAAGCA AGCTGATTTA TGATAACTGC TTTAAACTTC
AACAACCAGT AAGTCTTCAA GTGGAATTTA TTATTGATTC
TTTTATGTTA ATTTGTTAGG TCAAAAGAAA AATCTTTAGA
GCAAAATAAA AGTTTTGCTC TTTATTAGGA GGTTCTTTAG
ATATTACACT TTTAATTGGG TAGCTTATTT GCATGTATTT
TGAAACTATC TAAAGTAAAT AGTGTTTCCT TTGTATGCTT
ATCTTTAGCT AATGTGTTTT TTTTTTTGGT TTTAAAATAA
TGCTTCTAGT GAAAAAAATC ACAAAAACCT CAACACTGTA
ACGTTTGAGA GCAACGGCTA TTCAGTTCGG TTAAACCGAA

SNP_{—}1445 is in an untranslated region of AGTR2 mRNA (see TABLE 2B).

The nucleotide sequence of SNP 4795 (SEQ ID NO:3) is as follows:

ccaacacaaa agcacagcag ttgagaactg ggaaagcatc
gcactacaac tgctactgcc attaaccaca ttgtcctgga
tgcccaagag cttaagagcc cacttaccta cctggtacac
tgctactaca actgacatct gagaaagcca cccaaaggaa
caagaatttc cctgtctgga accaacagaa ttgtcactat/R
ttctgtacca gatcccaagg atacacatgc ttagcttact
attactacca ctgaaacttg caaaagaacc catcaagcat
tccattcccc agcacaaatt catcagtttc tatcaataac
ctcacaatgc cacacagagg aatagacaga tactactaag
gctgtttata gccaatgaaa tcatacacag tcttcacca

SNP_{—}4795 is in the AGTR2 genomic region (see TABLE 2B).

It is to be appreciated that certain aspects, modes, embodiments, variations and features of the invention are described below in various levels of detail in order to provide a substantial understanding of the invention. In general, such disclosure provides new uses of polynucleotide variations, SNPs, useful in the diagnosis and treatment of subjects in need thereof. Accordingly, the various aspects of the present invention relate to polynucleotides encoding the polynucleotide variations of the invention in the ACE and AGTR2 genes. The various aspects of the present invention further relate to diagnostic/theranostic methods and kits that use the polynucleotide variations of the invention to identify individuals predisposed to disease or to classify individuals with regard to drug responsiveness, side effects, or optimal drug dose. In other aspects, the invention provides methods for compound validation and a computer system for storing and analyzing data related to the polynucleotide variations of the invention. Accordingly, various particular embodiments that illustrate these aspects follow.

Definitions. The definitions of certain terms as used in this specification are provided below. Definitions of other terms may be found in the glossary provided by the U.S. Department of Energy, Office of Science, Human Genome Project <http://www.ornl.gov/sci/techresources/Human_Genome/glossary/>. A medical definition is provided by Chobanian et al., “JNC-7—Complete Version” Hypertension 42: 1206-1252 (2003). The American Heart Association's definition of hypertension may be found at the website <http://www.americanheart.org/presenter.jhtml?identifier=4623>. All of these references are hereby incorporated by reference.

As used herein, the term “allele” means a particular form of a gene or DNA sequence at a specific chromosomal location (locus).

As used herein, the term “antibody” includes, but is not limited to, polyclonal antibodies, monoclonal antibodies, humanized or chimaeric antibodies and biologically functional antibody fragments sufficient for binding of the antibody fragment to the protein.

As used herein, the term “clinical response” means any or all of the following: a quantitative measure of the response, no response, and adverse response (i.e., side effects).

As used herein, the term “clinical trial” means any research study designed to collect clinical data on responses to a particular treatment, and includes but is not limited to phase I, phase II and phase III clinical trials. Standard methods are used to define the patient population and to enrol subjects.

As used herein, the term “effective amount” of a compound is a quantity sufficient to achieve a desired therapeutic and/or prophylactic effect, for example, an amount which results in the prevention of or a decrease in the symptoms associated with hypertension. In a preferred embodiment, the compound is aliskiren.

The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds. In a preferred embodiment, the effective amount is aliskiren at 75, 150, or 300 mg administered once daily.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pairs found at one or more polymorphic sites in a locus on a pair of homologous chromosomes in an individual. As used herein, genotype includes a full-genotype and/or a sub-genotype.

As used herein, the term “locus” means a location on a chromosome or DNA molecule corresponding to a gene or a physical or phenotypic feature.

As used herein, the term “ACE modulating agent” or “AGTR2 modulating agent” is any compound that alters (e.g., increases or decreases) the expression level or biological activity level of ACE polypeptide or AGTR2 polypeptide, respectively, compared to the expression level or biological activity level of polypeptides in the absence of the modulating agent. The modulating agent can be a small molecule, polypeptide, carbohydrate, lipid, nucleotide, or combination thereof. The modulating agent may be an organic compound or an inorganic compound.

As used herein, the term “mutant” means any heritable variation from the wild-type that is the result of a mutation, e.g., single nucleotide polymorphism. The term “mutant” is used interchangeably with the terms “marker”, “biomarker”, and “target” throughout the specification.

As used herein, the term “medical condition” includes, but is not limited to, any condition or disease manifested as one or more physical and/or psychological symptoms for which treatment is desirable, and includes previously and newly identified diseases and other disorders. In a preferred embodiment, the medical condition is hypertension.

As used herein, the term “nucleotide pair” means the nucleotides found at a polymorphic site on the two copies of a chromosome from an individual.

As used herein, the term “polymorphic site” means a position within a locus at which at least two alternative sequences are found in a population, the most frequent of which has a frequency of no more than 99%.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “SNP nucleic acid” means a nucleic acid sequence, which comprises a nucleotide that is variable within an otherwise identical nucleotide sequence between individuals or groups of individuals, thus existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning. The SNP nucleic acids are referred to hereafter simply as “SNPs”. A SNP is the occurrence of nucleotide variability at a single position in the genome, in which two alternative bases occur at appreciable frequency (i.e., >1%) in the human population. A SNP may occur within a gene or within intergenic regions of the genome. SNP probes according to the invention are oligonucleotides that are complementary to a SNP nucleic acid.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., monkey (e.g., cynmologous monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

Identification and Characterization of Gene Sequence Variation. Due to their prevalence and widespread nature, SNPs have the potential to be important tools for locating genes that are involved in human disease conditions. See e.g., Wang et al., Science 280: 1077-1082 (1998). It is increasingly clear that the risk of developing many common disorders and the metabolism of medications used to treat these conditions are substantially influenced by underlying genomic variations, although the effects of any one variant might be small.

A SNP is said to be “allelic” in that due to the existence of the polymorphism, some members of a species may have an unmutated sequence (i.e., the original allele) whereas other members may have a mutated sequence (i.e., the variant or mutant allele).

An association between a SNP and a particular phenotype does not necessarily indicate or require that the SNP is causative of the phenotype. Instead, the association may merely be due to genome proximity between a SNP and those genetic factors actually responsible for a given phenotype, such that the SNP and said genetic factors are closely linked. That is, a SNP may be in linkage disequilibrium (“LD”) with the “true” functional variant. LD (a.k.a., allelic association) exists when alleles at two distinct locations of the genome are more highly associated than expected. Thus, a SNP may serve as a marker that has value by virtue of its proximity to a mutation that causes a particular phenotype.

In describing the polymorphic sites of the invention, reference is made to the sense strand of the gene for convenience. As recognized by the skilled artisan, however, nucleic acid molecules containing the gene may be complementary double stranded molecules and thus reference to a particular site on the sense strand refers as well to the corresponding site on the complementary antisense strand. That is, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the genomic variants described herein.

Identification and Characterization of SNPs. Many different techniques can be used to identify and characterize SNPs, including single-strand conformation polymorphism (SSCP) analysis, heteroduplex analysis by denaturing high-performance liquid chromatography (DHPLC) and direct DNA sequencing and computational methods. Shi et al., Clin. Chem. 47:164-172 (2001). There is a wealth of sequence information in public databases.

The most common SNP-typing methods currently include hybridization, primer extension, and cleavage methods. Each of these methods must be connected to an appropriate detection system. Detection technologies include fluorescent polarization (Chan et al., Genome Res. 9:492-499 (1999)), luminometric detection of pyrophosphate release (pyrosequencing) (Ahmadiian et al., Anal. Biochem. 280:103-10 (2000)), fluorescence resonance energy transfer (FRET)-based cleavage assays, DHPLC, and mass spectrometry (Shi, Clin. Chem. 47:164-172 (2001); U.S. Pat. No. 6,300,076 B1). Other methods of detecting and characterizing SNPs are those disclosed in U.S. Pat. Nos. 6,297,018 and 6,300,063.

Polymorphisms can also be detected using commercially available products, such as INVADER™ technology (available from Third Wave Technologies Inc. Madison, Wis., USA). In this assay, a specific upstream “invader” oligonucleotide and a partially overlapping downstream probe together form a specific structure when bound to complementary DNA template. This structure is recognized and cut at a specific site by the Cleavase enzyme, resulting in the release of the 5′ flap of the probe oligonucleotide. This fragment then serves as the “invader” oligonucleotide with respect to synthetic secondary targets and secondary fluorescently labelled signal probes contained in the reaction mixture. See also, Ryan D et al., Molecular Diagnosis 4(2): 135-144 (1999) and Lyamichev V et al., Nature Biotechnology 17: 292-296 (1999), see also U.S. Pat. Nos. 5,846,717 and 6,001,567.

The identity of polymorphisms may also be determined using a mismatch detection technique including, but not limited to, the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich P, Ann Rev Genet. 25:229-253 (1991)). Alternatively, variant alleles can be identified by single strand conformation polymorphism (SSCP) analysis (Orita et al., Genomics 5:874-879 (1989); Humphries et al., in Molecular Diagnosis of Genetic Diseases, Elles R, ed., pp. 321-340 (1996)) or denaturing gradient gel electrophoresis (DGGE) (Wartell et al., Nucl. Acids. Res. 18:2699-2706 (1990); Sheffield et al., Proc. Natl. Acad. Sci. USA 86:232-236 (1989)). A polymerase-mediated primer extension method may also be used to identify the polymorphisms. Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, and U.S. Pat. Nos. 5,302,509 and 5,945,283. Extended primers containing a polymorphism may be detected by mass spectrometry as described in U.S. Pat. No. 5,605,798. Another primer extension method is allele-specific PCR (Ruafio et al., Nucl. Acids. Res. 17:8392 (1989); Ruafio et al., Nucl. Acids. Res. 19: 6877-6882 (1991); WO 93/22456; Turki et al., J. Clin. Invest. 95:1635-1641 (1995)). In addition, multiple polymorphic sites may be investigated by simultaneously amplifying multiple regions of the nucleic acid using sets of allele-specific primers as described in published PCT patent application WO 89/10414.

Haplotyping and Genotyping Oligonucleotides. The invention provides methods and compositions for haplotyping and/or genotyping the gene in an individual. As used herein, the terms “genotype” and “haplotype” mean the genotype or haplotype containing the nucleotide pair or nucleotide, respectively, that is present at one or more of the novel polymorphic sites described herein and may optionally also include the nucleotide pair or nucleotide present at one or more additional polymorphic sites in the gene. The additional polymorphic sites may be currently known polymorphic sites or sites that are subsequently discovered.

The compositions of the invention contain oligonucleotide probes and primers designed to specifically hybridize to one or more target regions containing, or that are adjacent to, a polymorphic site. Oligonucleotide compositions of the invention are useful in methods for genotyping and/or haplotyping a gene in an individual. The methods and compositions for establishing the genotype or haplotype of an individual at the novel polymorphic sites described herein are useful for studying the effect of the polymorphisms in the aetiology of diseases affected by the expression and function of the protein, studying the efficacy of drugs targeting, predicting individual susceptibility to diseases affected by the expression and function of the protein and predicting individual responsiveness to drugs targeting the gene product.

Genotyping oligonucleotides of the invention may be immobilized on or synthesized on a solid surface such as a microchip, bead, or glass slide. See, e.g., WO 98/20020 and WO 98/20019.

Genotyping oligonucleotides may hybridize to a target region located one to several nucleotides downstream of one of the novel polymorphic sites identified herein. Such oligonucleotides are useful in polymerase-mediated primer extension methods for detecting one of the novel polymorphisms described herein and therefore such genotyping oligonucleotides are referred to herein as “primer-extension oligonucleotides”.

Direct Genotyping Method of the Invention. A genotyping method of the invention may involve isolating from an individual a nucleic acid mixture comprising the two copies of a gene of interest or fragment thereof, and determining the identity of the nucleotide pair at one or more of the polymorphic sites in the two copies. As will be readily understood by the skilled artisan, the two “copies” of a gene in an individual may be the same allele or may be different alleles. In a particularly preferred embodiment, the genotyping method comprises determining the identity of the nucleotide pair at each polymorphic site. Typically, the nucleic acid mixture is isolated from a biological sample taken from the individual, such as a blood sample or tissue sample. Suitable tissue samples include whole blood, semen, saliva, tears, urine, fecal material, sweat, buccal smears, skin and hair.

In the EXAMPLE below, single nucleotide polymorphism (SNP) assays probe sets for the genotyping assay were generated for ABI's Assays-by-Design® platform. Livak K J, Marmaro J & Todd J A, Nature Genetics 9: 341-2 (1995). Genotyping was performed on 10 ng of genomic DNA according to the manufacturer's instructions.

Direct Haplotyping Method of the Invention. A haplotyping method of the invention may include isolating from an individual a nucleic acid molecule containing only one of the two copies of a gene of interest, or a fragment thereof, and determining the identity of the nucleotide at one or more of the polymorphic sites in that copy. Direct haplotyping methods include, for example, CLASPER System™ technology (U.S. Pat. No. 5,866,404) or allele-specific long-range PCR (Michalotos-Beloin et al., Nucl. Acids. Res. 24: 4841-4843 (1996)). The nucleic acid may be isolated using any method capable of separating the two copies of the gene or fragment. As will be readily appreciated by those skilled in the art, any individual clone will only provide haplotype information on one of the two gene copies present in an individual. In one embodiment, a haplotype pair is determined for an individual by identifying the phased sequence of nucleotides at one or more of the polymorphic sites in each copy of the gene that is present in the individual. In a preferred embodiment, the haplotyping method comprises identifying the phased sequence of nucleotides at each polymorphic site in each copy of the gene.

In both the genotyping and haplotyping methods, the identity of a nucleotide (or nucleotide pair) at a polymorphic site may be determined by amplifying a target regions containing the polymorphic sites directly from one or both copies of the gene, or fragments thereof, and sequencing the amplified regions by conventional methods. The genotype or haplotype for the gene of an individual may also be determined by hybridization of a nucleic sample containing one or both copies of the gene to nucleic acid arrays and subarrays such as described in WO 95/11995.

Indirect Genotyping Method using Polymorphic Sites in Linkage Disequilibrium with a Target Polymorphism. In addition, the identity of the alleles present at any of the novel polymorphic sites of the invention may be indirectly determined by genotyping other polymorphic sites in linkage disequilibrium with those sites of interest. As described above, two sites are said to be in linkage disequilibrium if the presence of a particular variant at one site is indicative of the presence of another variant at a second site. Stevens J C, Mol. Diag. 4: 309-317 (1999). Polymorphic sites in linkage disequilibrium with the polymorphic sites of the invention may be located in regions of the same gene or in other genomic regions.

Amplifying a Target Gene Region. The target regions may be amplified using any oligonucleotide-directed amplification method, including but not limited to polymerase chain reaction (PCR). (U.S. Pat. No. 4,965,188), ligase chain reaction (LCR) (Barany et al., Proc. Natl. Acad. Sci. USA 88:189-193 (1991); published PCT patent application WO 90/01069), and oligonucleotide ligation assay (OLA) (Landegren et al., Science 241: 1077-1080 (1988)). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, published PCT patent application WO 89/06700) and isothermal methods (Walker et al., Proc. Natl. Acad. Sci. USA 89:392-396 (1992)).

Hybridizing Allele-Specific Oligonucleotide to a Target Gene. A polymorphism in the target region may be assayed before or after amplification using one of several hybridization-based methods known in the art. Typically, allele-specific oligonucleotides are utilized in performing such methods. The allele-specific oligonucleotides may be used as differently labelled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking, baking, etc. Allele-specific oligonucleotide may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibres, chips, dishes, and beads. The solid support may be treated, coated or derivatised to facilitate the immobilization of the allele-specific oligonucleotide or target nucleic acid.

Determining Population Genotypes and Haplotypes and Correlating Them with a Trait. The invention provides a method for determining the frequency of a genotype or haplotype in a population. The method comprises determining the genotype or the haplotype for a gene present in each member of the population, wherein the genotype or haplotype comprises the nucleotide pair or nucleotide detected at one or more of the polymorphic sites in the gene, and calculating the frequency at which the genotype or haplotype is found in the population. The population may be a reference population, a family population, a same sex population, a population group, or a trait population (e.g., a group of individuals exhibiting a trait of interest such as a medical condition or response to a therapeutic treatment).

In another aspect of the invention, frequency data for genotypes and/or haplotypes found in a reference population are used in a method for identifying an association between a trait and a genotype or a haplotype. The trait may be any detectable phenotype, including but not limited to susceptibility to a disease or response to a treatment. The method involves obtaining data on the frequency of the genotypes or haplotypes of interest in a reference population and comparing the data to the frequency of the genotypes or haplotypes in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

The frequency data for the reference and/or trait populations are obtained by accessing previously determined frequency data, which may be in written or electronic form. For example, the frequency data may be present in a database that is accessible by a computer. Once the frequency data are obtained, the frequencies of the genotypes or haplotypes of interest in the reference and trait populations are compared.

When polymorphisms are being analyzed, a calculation may be performed to correct for a significant association that might be found by chance. For statistical methods useful in the methods of the invention, see Statistical Methods in Biology, 3^{rd} edition, Bailey N T J, (Cambridge Univ. Press, 1997); Waterman M S, Introduction to Computational Biology (CRC Press, 2000) and Bioinformatics, Baxevanis A D & Ouellette B F F editors (John Wiley & Sons, Inc., 2001).

In another embodiment, the haplotype frequency data for different groups are examined to determine whether they are consistent with Hardy-Weinberg equilibrium. Hartl D L et al., Principles of Population Genomics, 3rd Ed. (Sinauer Associates, Sunderland, Mass., 1997).

In another embodiment, statistical analysis is performed by the use of standard ANOVA tests with a Bonferoni correction or a bootstrapping method that simulates the genotype phenotype correlation many times and calculates a significance value. ANOVA is used to test hypotheses about whether a response variable is caused by or correlates with one or more traits or variables that can be measured. Fisher L D & vanBelle G, Biostatistics: A Methodology for the Health Sciences (Wiley-Interscience, New York, 1993) Ch. 10.

In one embodiment for predicting a haplotype pair, the analysis includes an assigning step, as follows: First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair.

In another embodiment, a detectable genotype or haplotype that is in linkage disequilibrium with a genotype or haplotype of interest may be used as a surrogate marker. A genotype that is in linkage disequilibrium with another genotype is indicated where a particular genotype or haplotype for a given gene is more frequent in the population that also demonstrates the potential surrogate marker genotype than in the reference population. If the frequency is statistically significant, then the marker genotype is predictive of that genotype or haplotype, and can be used as a surrogate marker.

Another method for finding correlations between haplotype content and clinical responses uses predictive models based on error-minimizing optimization algorithms, one of which is a genetic algorithm. See, Judson R, “Genetic Algorithms and Their Uses in Chemistry” in Reviews in Computational Chemistry, Ch. 10, Lipkowitz K B & Boyd D B, eds. (VCH Publishers, New York, 1997) pp. 1-73. Simulated annealing (Press et al., Numerical Recipes in C: The Art of Scientific Computing, Ch. 10 (Cambridge University Press, Cambridge, 1992), neural networks (Rich E & Knight K, Artificial Intelligence, 2nd Edition, Ch. 10 (McGraw-Hill, New York, 1991), standard gradient descent methods (Press et al., supra Ch. 10), or other global or local optimization approaches (see discussion in Judson, supra) can also be used.

In the EXAMPLE below, genotype-phenotype association studies and related analyses were performed in SAS (Cary, N.C., USA) using Analyst®. Association tests used categorical genotypes as the independent variable, with no assumption about dominance, and the various efficacy variables as dependent variables. Tests of continuous dependent variables used an ANCOVA analysis, and logistic regression was used for categorical dependent variables. Covariates in the genotype-phenotype association analysis were: treatment, trial region and baseline measurement. ANCOVA analysis was repeated with the same model for each treatment group. In addition, the percent of responders was analyzed by means of a logistic regression model with treatment and region as factors and baseline as a covariate. Associations with p<0.05 in the whole dataset were deemed significant.

Correlating Subject Genotype or Haplotype to Treatment Response. In preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome, including efficacy measurements, pharmacokinetic measurements and side-effect measurements.

In another preferred embodiment, the trait of interest is a clinical response exhibited by a patient to some therapeutic treatment, for example, response to a drug targeting or to a therapeutic treatment for a medical condition.

To deduce a correlation between a clinical response to a treatment and a genotype or haplotype, genotype or haplotype data is obtained on the clinical responses exhibited by a population of individuals who received the treatment, hereinafter the “clinical population”. This clinical data may be obtained by analyzing the results of a clinical trial that has already been run and/or by designing and carrying out one or more new clinical trials.

The individuals included in the clinical population are usually graded for the existence of the medical condition of interest. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population, and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the gene for each individual in the trial population is genotyped and/or haplotyped, which may be done before or after administering the treatment.

These results are then analyzed to determine if any observed variation in clinical response between polymorphism groups is statistically significant. Statistical analysis methods, which may be used, are described in Fisher L D & vanBelle G, Biostatistics: A Methodology for the Health Sciences (Wiley-Interscience, New York, 1993). This analysis may also include a regression calculation of which polymorphic sites in the gene contribute most significantly to the differences in phenotype.

After both the clinical and polymorphism data have been obtained, correlations between individual response and genotype or haplotype content are created. Correlations may be produced in several ways. In one method, individuals are grouped by their genotype or haplotype (or haplotype pair) (also referred to as a polymorphism group), and then the averages and standard deviations of clinical responses exhibited by the members of each polymorphism group are calculated.

From the analyses described above, the skilled artisan that predicts clinical response as a function of genotype or haplotype content may readily construct a mathematical model. The identification of an association between a clinical response and a genotype or haplotype (or haplotype pair) for the gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the gene), a serological test, or a physical exam measurement. The only requirement is that there be a good correlation between the diagnostic test results and the underlying genotype or haplotype. In a preferred embodiment, this diagnostic method uses the predictive haplotyping method described above.

Assigning a Subject to a Genotype Group. As one of skill in the art will understand, there will be a certain degree of uncertainty involved in making this determination. Therefore, the standard deviations of the control group levels would be used to make a probabilistic determination and the methods of this invention would be applicable over a wide range of probability based genotype group determinations. Thus, for example and not by way of limitation, in one embodiment, if the measured level of the gene expression product falls within 2.5 standard deviations of the mean of any of the control groups, then that individual may be assigned to that genotype group. In another embodiment if the measured level of the gene expression product falls within 2.0 standard deviations of the mean of any of the control groups then that individual may be assigned to that genotype group. In still another embodiment, if the measured level of the gene expression product falls within 1.5 standard deviations of the mean of any of the control groups then that individual may be assigned to that genotype group. In yet another embodiment, if the measured level of the gene expression product is 1.0 or less standard deviations of the mean of any of the control groups levels then that individual may be assigned to that genotype group.

Thus this process allows determination, with various degrees of probability, which group a specific subject should be placed in, and such assignment to a genotype group would then determine the risk category into which the individual should be placed.

Correlation between Clinical Response and Genotype or Haplotype. In order to deduce a correlation between clinical response to a treatment and a genotype or haplotype, it is necessary to obtain data on the clinical responses exhibited by a population of individuals who received the treatment, hereinafter the “clinical population.” This clinical data may be obtained by analyzing the results of a clinical trial that has already been run and/or the clinical data may be obtained by designing and carrying out one or more new clinical trials.

The standard control levels of the gene expression product, thus determined in the different control groups, would then be compared with the measured level of a gene expression product in a given patient. This gene expression product could be the characteristic mRNA associated with that particular genotype group or the polypeptide gene expression product of that genotype group. The patient could then be classified or assigned to a particular genotype group based on how similar the measured levels were compared to the control levels for a given group.

Computer System for Storing or Displaying Polymorphism Data. The invention also provides a computer system for storing and displaying polymorphism data determined for the gene. The computer system comprises a computer processing unit, a display, and a database containing the polymorphism data. The polymorphism data includes the polymorphisms, the genotypes and the haplotypes identified for a given gene in a reference population. In a preferred embodiment, the computer system is capable of producing a display showing haplotypes organized according to their evolutionary relationships. A computer may implement any or all analytical and mathematical operations involved in practicing the methods of the present invention. In addition, the computer may execute a program that generates views (or screens) displayed on a display device and with which the user can interact to view and analyze large amounts of information relating to the gene and its genomic variation, including chromosome location, gene structure, and gene family, gene expression data, polymorphism data, genetic sequence data, and clinical population data (e.g., data on ethnogeographic origin, clinical responses, genotypes, and haplotypes for one or more populations). The polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

Nucleic Acid-based Diagnostics. In another aspect, the invention provides SNP probes, which are useful in classifying subjects according to their types of genetic variation. The SNP probes according to the invention are oligonucleotides, which discriminate between SNPs in conventional allelic discrimination assays. In certain preferred embodiments, the oligonucleotides according to this aspect of the invention are complementary to one allele of the SNP nucleic acid, but not to any other allele of the SNP nucleic acid. Oligonucleotides according to this embodiment of the invention can discriminate between SNPs in various ways. For example, under stringent hybridization conditions, an oligonucleotide of appropriate length will hybridize to one SNP, but not to any other. The oligonucleotide may be labelled using a radiolabel or a fluorescent molecular tag. Alternatively, an oligonucleotide of appropriate length can be used as a primer for PCR, wherein the 3′ terminal nucleotide is complementary to one allele containing a SNP, but not to any other allele. In this embodiment, the presence or absence of amplification by PCR determines the haplotype of the SNP.

Genomic and cDNA fragments of the invention comprise at least one novel polymorphic site identified herein, have a length of at least 10 nucleotides, and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

Kits of the Invention. The invention provides nucleic acid and polypeptide detection kits useful for haplotyping and/or genotyping the gene in an individual. Such kits are useful for classifying individuals for the purpose of classifying individuals. Specifically, the invention encompasses kits for detecting the presence of a polypeptide or nucleic acid corresponding to a marker of the invention in a biological sample, e.g., any bodily fluid including, but not limited to, serum, plasma, lymph, cystic fluid, urine, stool, cerebrospinal fluid, ascities fluid or blood, and including biopsy samples of body tissue. For example, the kit can comprise a labelled compound or agent capable of detecting a polypeptide or an mRNA encoding a polypeptide corresponding to a marker of the invention in a biological sample and means for determining the amount of the polypeptide or mRNA in the sample, e.g., an antibody which binds the polypeptide or an oligonucleotide probe which binds to DNA or mRNA encoding the polypeptide. Kits can also include instructions for interpreting the results obtained using the kit.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as in the case of PCR. In a preferred embodiment, such kit may further comprise a DNA sample collecting means.

For antibody-based kits, the kit can comprise, e.g., (1) a first antibody, e.g., attached to a solid support, which binds to a polypeptide corresponding to a marker or the invention; and, optionally (2) a second, different antibody which binds to either the polypeptide or the first antibody and is conjugated to a detectable label.

For oligonucleotide-based kits, the kit can comprise, e.g., (1) an oligonucleotide, e.g., a detectably-labelled oligonucleotide, which hybridizes to a nucleic acid sequence encoding a polypeptide corresponding to a marker of the invention; or (2) a pair of primers useful for amplifying a nucleic acid molecule corresponding to a marker of the invention.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

Nucleic Acid Sequences of the Invention. In one aspect, the invention comprises one or more isolated polynucleotides. The invention also encompasses allelic variants of the same, that is, naturally occurring alternative forms of the isolated polynucleotides that encode mutant polypeptides that are identical, homologous or related to those encoded by the polynucleotides. Alternatively, non-naturally occurring variants may be produced by mutagenesis techniques or by direct synthesis techniques well-known in the art.

Accordingly, nucleic acid sequences capable of hybridizing at low stringency with any nucleic acid sequences encoding mutant polypeptide of the present invention are considered to be within the scope of the invention. Standard stringency conditions are well characterized in standard molecular biology cloning texts. See, for example Molecular Cloning A Laboratory Manual, 2nd Ed., Sambrook, Fritsch & Maniatis (Cold Spring Harbor Laboratory Press, 1989); DNA Cloning, Volumes I and II, Glover D N, ed. (1985); Oligonucleotide Synthesis, Gait M J, ed. (1984); Nucleic Acid Hybridization, Hames B D & Higgins S J, eds. (1984).

Characterizing Gene Expression Level. Methods to detect and measure mRNA levels (i.e., gene transcription level) and levels of polypeptide gene expression products (i.e., gene translation level) are well-known in the art and include the use of nucleotide microarrays and polypeptide detection methods involving mass spectrometers and/or antibody detection and quantification techniques. See also, Strachan T & Read A, Human Molecular Genetics, 2^{nd} Edition. (John Wiley and Sons, Inc. Publication, New York, 1999).

Determination of Target Gene Transcription. The determination of the level of the expression product of the gene in a biological sample, e.g., the tissue or body fluids of an individual, may be performed in a variety of ways. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells. See, e.g., Ausubel et al., ed., Curr. Prot. Mol. Biol. (John Wiley & Sons, New York, 1987-1999).

In one embodiment, the level of the mRNA expression product of the target gene is determined. Methods to measure the level of a specific mRNA are well-known in the art and include Northern blot analysis, reverse transcription PCR and real time quantitative PCR or by hybridization to a oligonucleotide array or microarray. In other more preferred embodiments, the determination of the level of expression may be performed by determination of the level of the protein or polypeptide expression product of the gene in body fluids or tissue samples including but not limited to blood or serum. Large numbers of tissue samples can readily be processed using techniques well-known to those of skill in the art, such as, e.g., the single-step RNA isolation process of U.S. Pat. No. 4,843,155.

The isolated mRNA can be used in hybridization or amplification assays that include, but are not limited to, Southern or Northern analyses, PCR analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a marker of the present invention. Other suitable probes for use in the diagnostic assays of the invention are described herein. Hybridization of an mRNA with the probe indicates that the marker in question is being expressed.

In one format, the probes are immobilized on a solid surface and the mRNA is contacted with the probes, for example, in an Affymetrix gene chip array (Affymetrix, Calif. USA). A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in U.S. Pat. No. 4,683,202); ligase chain reaction (Barany et al., Proc. Natl. Acad. Sci. USA 88:189-193 (1991)) self-sustained sequence replication (Guatelli et al., Proc. Natl. Acad. Sci. USA 87: 1874-1878 (1990)); transcriptional amplification system (Kwoh et al., Proc. Natl. Acad. Sci. USA 86: 1173-1177 (1989)); Q-Beta Replicase (Lizardi et al., Biol. Technology 6: 1197 (1988)); rolling circle replication (U.S. Pat. No. 5,854,033); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, “amplification primers” are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length.

Real-time quantitative PCR (RT-PCR) is one way to assess gene expression levels, e.g., of genes of the invention, e.g., those containing SNPs and polymorphisms of interest. The RT-PCR assay utilizes an RNA reverse transcriptase to catalyze the synthesis of a DNA strand from an RNA strand, including an mRNA strand. The resultant DNA may be specifically detected and quantified and this process may be used to determine the levels of specific species of mRNA. One method for doing this is TAQMAN® (PE Applied Biosystems, Foster City, Calif., USA) and exploits the 5′ nuclease activity of AMPLITAQ GOLD™ DNA polymerase to cleave a specific form of probe during a PCR reaction. This is referred to as a TAQMAN™ probe. See Luthra et al., Am. J. Pathol. 153: 63-68 (1998); Kuimelis et al., Nucl. Acids Symp. Ser. 37: 255-256 (1997); and Mullah et al., Nucl. Acids Res. 26(4): 1026-1031 (1998)). During the reaction, cleavage of the probe separates a reporter dye and a quencher dye, resulting in increased fluorescence of the reporter. The accumulation of PCR products is detected directly by monitoring the increase in fluorescence of the reporter dye. Heid et al., Genome Res. 6(6): 986-994 (1996)). The higher the starting copy number of nucleic acid target, the sooner a significant increase in fluorescence is observed. See Gibson, Heid & Williams et al., Genome Res. 6: 995-1001 (1996).

Other technologies for measuring the transcriptional state of a cell produce pools of restriction fragments of limited complexity for electrophoretic analysis, such as methods combining double restriction enzyme digestion with phasing primers (see, e.g., EP 0 534858 A1), or methods selecting restriction fragments with sites closest to a defined mRNA end. (See, e.g., Prashar & Weissman, Proc. Natl. Acad, Sci. USA 93(2) 659-663 (1996)).

Other methods statistically sample cDNA pools, such as by sequencing sufficient bases, e.g., 20-50 bases, in each of multiple cDNAs to identify each cDNA, or by sequencing short tags, e.g., 9-10 bases, which are generated at known positions relative to a defined mRNA end pathway pattern. See, e.g., Velculescu, Science 270: 484-487 (1995). The cDNA levels in the samples are quantified and the mean, average and standard deviation of each cDNA is determined using by standard statistical means well-known to those of skill in the art. Norman T. J. Bailey, Statistical Methods In Biology, 3rd Edition (Cambridge University Press, 1995).

Detection of Polypeptides. Immunological Detection Methods. Expression of the protein encoded by the genes of the invention can be detected by a probe which is detectably labelled, or which can be subsequently labelled. The term “labelled”, with regard to the probe or antibody, is intended to encompass direct-labelling of the probe or antibody by coupling, i.e., physically linking, a detectable substance to the probe or antibody, as well as indirect-labelling of the probe or antibody by reactivity with another reagent that is directly-labelled. Examples of indirect labelling include detection of a primary antibody using a fluorescently-labelled secondary antibody and end-labelling of a DNA probe with biotin such that it can be detected with fluorescently-labelled streptavidin. Generally, the probe is an antibody that recognizes the expressed protein. A variety of formats can be employed to determine whether a sample contains a target protein that binds to a given antibody. Immunoassay methods useful in the detection of target polypeptides of the present invention include, but are not limited to, e.g., dot blotting, western blotting, protein chips, competitive and non-competitive protein binding assays, enzyme-linked immunosorbant assays (ELISA), immunohistochemistry, fluorescence activated cell sorting (FACS), and others commonly used and widely-described in scientific and patent literature, and many employed commercially. A skilled artisan can readily adapt known protein/antibody detection methods for use in determining whether cells express a marker of the present invention and the relative concentration of that specific polypeptide expression product in blood or other body tissues. Proteins from individuals can be isolated using techniques that are well-known to those of skill in the art. The protein isolation methods employed can, e.g., be such as those described in Harlow & Lane, Antibodies: A Laboratory Manual (Cold Spring Harbor Laboratory Press, Cold Spring Harbor, N.Y., 1988)).

For the production of antibodies to a protein encoded by one of the disclosed genes, various host animals may be immunized by injection with the polypeptide, or a portion thereof. Such host animals may include, but are not limited to, rabbits, mice and rats. Various adjuvants may be used to increase the immunological response, depending on the host species including, but not limited to, Freund's (complete and incomplete), mineral gels, such as aluminium hydroxide; surface active substances, such as lysolecithin, pluronic polyols, polyanions, peptides, oil emulsions, keyhole limpet haemocyanin and dinitrophenol; and potentially useful human adjuvants, such as bacille Camette-Guerin (BCG) and Corynebacterium parvum.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler & Milstein, Nature 256: 495-497 (1975); and U.S. Pat. No. 4,376,110; the human B-cell hybridoma technique of Kosbor et al., Immunol. Today 4: 72 (1983); Cole et al., Proc. Natl. Acad. Sci. USA 80: 2026-2030 (1983); and the EBV-hybridoma technique of Cole et al., Monoclonal Antibodies and Cancer Therapy (Alan R. Liss, Inc., 1985) pp. 77-96.

In addition, techniques developed for the production of “chimaeric antibodies” (see Morrison et al., Proc. Natl. Acad. Sci. USA 81: 6851-6855 (1984); Neuberger et al., Nature 312: 604-608 (1984); and Takeda et al., Nature 314: 452-454 (1985)), by splicing the genes from a mouse antibody molecule of appropriate antigen specificity together with genes from a human antibody molecule of appropriate biological activity can be used. A chimaeric antibody is a molecule in which different portions are derived from different animal species, such as those having a variable or hypervariable region derived form a murine mAb and a human immunoglobulin constant region.

Alternatively, techniques described for the production of single chain antibodies (U.S. Pat. No. 4,946,778; Bird, Science 242: 423-426 (1988); Huston et al., Proc. Natl. Acad. Sci. USA 85: 5879-5883 (1988); and Ward et al., Nature 334: 544-546 (1989)) can be adapted to produce differentially expressed gene single-chain antibodies.

Techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibodies or antibody fragments can be used in methods, such as Western blots or immunofluorescence techniques, to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

A useful method, for ease of detection, is the sandwich ELISA, of which a number of variations exist, all of which are intended to be used in the methods and assays of the present invention. As used herein, “sandwich assay” is intended to encompass all variations on the basic two-site technique. Immunofluorescence and EIA techniques are both very well-established in the art. However, other reporter molecules, such as radioisotopes, chemiluminescent or bioluminescent molecules may also be employed. It will be readily apparent to the skilled artisan how to vary the procedure to suit the required use.

Whole genome monitoring of protein, i.e., the “proteome,” can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. As noted above, methods for making monoclonal antibodies are well-known. See, e.g., Harlow & Lane, Antibodies: A Laboratory Manual” (Cold Spring Harbor Laboratory Press, Cold Spring Harbor, N.Y., 1988). In a preferred embodiment, monoclonal antibodies are raised against synthetic peptide fragments designed based on genomic sequence of the cell. With such an antibody array, proteins from the cell are contacted to the array and their binding is measured with assays known in the art.

Detection of Polypeptides. Two-Dimensional Gel Electrophoresis. Two-dimensional gel electrophoresis is well-known in the art and typically involves isoelectric focusing along a first dimension followed by SDS-PAGE electrophoresis along a second dimension. See, e.g., Hames et al., Gel Electrophoresis of Proteins: A Practical Approach (IRL Press, New York, 1990); Shevchenko et al., Proc. Natl. Acad. Sci. USA 93: 14440-14445 (1996); Sagliocco et al., Yeast 12: 1519-1533 (1996); and Lander, Science 274: 536-539 (1996).

Detection of Polypeptides. Mass Spectroscopy. The identity as well as expression level of target polypeptide can be determined using mass spectrosopy technique (MS). MS-based analysis methodology is useful for analysis of isolated target polypeptide as well as analysis of target polypeptide in a biological sample. MS formats for use in analyzing a target polypeptide include ionization (I) techniques, such as, but not limited to, matrix assisted laser desorption (MALDI), continuous or pulsed electrospray ionization (ESI) and related methods, such as ionspray or thermospray, and massive cluster impact (MCI). Such ion sources can be matched with detection formats, including linear or non-linear reflectron time of flight (TOF), single or multiple quadrupole, single or multiple magnetic sector, Fourier transform ion cyclotron resonance (FTICR), ion trap and combinations thereof such as ion-trap/TOF. For ionization, numerous matrix/wavelength combinations (e.g., matrix assisted laser desorption (MALDI)) or solvent combinations (e.g., ESI) can be employed.

For mass spectroscopy (MS) analysis, the target polypeptide can be solubilised in an appropriate solution or reagent system. The selection of a solution or reagent system, e.g., an organic or inorganic solvent, will depend on the properties of the target polypeptide and the type of MS performed, and is based on methods well-known in the art. See, e.g., Vorm et al., Anal. Chem. 61: 3281 (1994) for MALDI; and Valaskovic et al., Anal. Chem. 67: 3802 (1995), for ESI. MS of peptides also is described, e.g., in International PCT Application No. WO 93/24834 and U.S. Pat. No. 5,792,664. A solvent is selected that minimizes the risk that the target polypeptide will be decomposed by the energy introduced for the vaporization process. A reduced risk of target polypeptide decomposition can be achieved, e.g., by embedding the sample in matrix. A suitable matrix can be an organic compound such as a sugar, e.g., a pentose or hexose, or a polysaccharide such as cellulose. Such compounds are decomposed thermolytically into CO₂ and H₂O such that no residues are formed that can lead to chemical reactions. The matrix also can be an inorganic compound, such as nitrate of ammonium, which is decomposed essentially without leaving any residue. Use of these and other solvents is known to those of skill in the art. See, e.g., U.S. Pat. No. 5,062,935. Electrospray MS has been described by Fenn et al., J. Phys. Chem. 88: 4451-4459 (1984); and PCT Application No. WO 90/14148; and current applications are summarized in review articles. See, Smith et al., Anal. Chem. 62: 882-89 (1990) and Ardrey, Spectroscopy 4: 10-18 (1992).

The mass of a target polypeptide determined by MS can be compared to the mass of a corresponding known polypeptide. For example, where the target polypeptide is a mutant protein, the corresponding known polypeptide can be the corresponding non-mutant protein, e.g., wild-type protein. With ESI, the determination of molecular weights in femtomole amounts of sample is very accurate due to the presence of multiple ion peaks, all of which can be used for mass calculation. Sub-attomole levels of protein have been detected, e.g., using ESI MS (Valaskovic et al., Science 273: 1199-1202 (1996)) and MALDI MS (Li et al., J. Am. Chem. Soc. 118: 1662-1663 (1996)).

Matrix Assisted Laser Desorption (MALDI). The level of the target protein in a biological sample, e.g., body fluid or tissue sample, may be measured by means of mass spectrometric (MS) methods including, but not limited to, those techniques known in the art as matrix-assisted laser desorption/ionization, time-of-flight mass spectrometry (MALDI-TOF-MS) and surfaces enhanced for laser desorption/ionization, time-of-flight mass spectrometry (SELDI-TOF-MS) as further detailed below. Methods for performing MALDI are well-known to those of skill in the art. See, e.g., Juhasz et al., Analysis, Anal. Chem. 68: 941-946 (1996), and see also, e.g., U.S. Pat. Nos. 5,777,325; 5,742,049; 5,654,545; 5,641,959; 5,654,545 and 5,760,393 for descriptions of MALDI and delayed extraction protocols. Numerous methods for improving resolution are also known. MALDI-TOF-MS has been described by Hillenkamp et al., Biological Mass Spectrometry, Burlingame & McCloskey, eds. (Elsevier Science Publ., Amsterdam, 1990) pp. 49-60.

A variety of techniques for marker detection using mass spectroscopy can be used. See Bordeaux Mass Spectrometry Conference Report, Hillenkamp, ed., pp. 354-362 (1988); Bordeaux Mass Spectrometry Conference Report, Karas & Hillenkamp, eds., pp. 416-417 (1988); Karas & Hillenkamp, Anal. Chem. 60: 2299-2301 (1988); and Karas et al., Biomed. Environ. Mass Spectrum 18: 841-843 (1989). The use of laser beams in TOF-MS is shown, e.g., in U.S. Pat. Nos. 4,694,167; 4,686,366, 4,295,046 and 5,045,694, which are incorporated herein by reference in their entireties. Other MS techniques allow the successful volatilization of high molecular weight biopolymers, without fragmentation, and have enabled a wide variety of biological macromolecules to be analyzed by mass spectrometry.

Surfaces Enhanced for Laser Desorption/Ionization (SELDI). Other techniques are used which employ new MS probe element compositions with surfaces that allow the probe element to actively participate in the capture and docking of specific analytes, described as Affinity Mass Spectrometry (AMS). See SELDI patents: U.S. Pat. Nos. 5,719,060; 5,894,063; 6,020,208; 6,027,942; 6,124,137; and published U.S. patent application No. U.S. 2003/0003465. Several types of new MS probe elements have been designed with Surfaces Enhanced for Affinity Capture (SEAC). See Hutchens & Yip, Rapid Commun. Mass Spectrom. 7: 576-580 (1993). SEAC probe elements have been used successfully to retrieve and tether different classes of biopolymers, particularly proteins, by exploiting what is known about protein surface structures and biospecific molecular recognition. The immobilized affinity capture devices on the MS probe element surface, i.e., SEAC, determines the location and affinity (specificity) of the analyte for the probe surface, therefore the subsequent analytical MS process is efficient.

Within the general category of SELDI are three separate subcategories: (1) Surfaces Enhanced for Neat Desorption (SEND), where the probe element surfaces, i.e., sample presenting means, are designed to contain Energy Absorbing Molecules (EAM) instead of “matrix” to facilitate desorption/ionizations of analytes added directly (neat) to the surface. (2) SEAC, where the probe element surfaces, i.e., sample presenting means, are designed to contain chemically defined and/or biologically defined affinity capture devices to facilitate either the specific or non-specific attachment or adsorption (so-called docking or tethering) of analytes to the probe surface, by a variety of mechanisms (mostly non-covalent). (3) Surfaces Enhanced for Photolabile Attachment and Release (SEPAR), where the probe element surfaces, i.e., sample presenting means, are designed or modified to contain one or more types of chemically defined cross-linking molecules to serve as covalent docking devices. The chemical specificities determining the type and number of the photolabile molecule attachment points between the SEPAR sample presenting means (i.e., probe element surface) and the analyte (e.g., protein) may involve any one or more of a number of different residues or chemical structures in the analyte (e.g., His, Lys, Arg, Tyr, Phe and Cys residues in the case of proteins and peptides).

Other Aspects of the Biological State. In various embodiments of the invention, aspects of the biological activity state, or mixed aspects can be measured in order to obtain drug and pathway responses. The activities of proteins relevant to the characterization of cell function can be measured, and embodiments of this invention can be based on such measurements. Activity measurements can be performed by any functional, biochemical or physical means appropriate to the particular activity being characterized. Where the activity involves a chemical transformation, the cellular protein can be contacted with natural substrates, and the rate of transformation measured. Where the activity involves association in multimeric units, e.g., association of an activated DNA binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the methods of this invention. In alternative and non-limiting embodiments, response data may be formed of mixed aspects of the biological state of a cell. Response data can be constructed from, e.g., changes in certain mRNA abundances, changes in certain protein abundances and changes in certain protein activities.

The following EXAMPLE is presented in order to more fully illustrate the preferred embodiments of the invention. This EXAMPLE should in no way be construed as limiting the scope of the invention, as defined by the appended claims.

### Example

### Comparison of Aliskiren to Placebo and Irbesartan in Patients With Mild-to-Moderate Essential Hypertension

Introduction and summary. A retrospective pharmacogenetic analysis was conducted in an attempt to evaluate potential association between genetic variation and clinical outcome in a clinical trial. Specifically, 48 polymorphisms were examined in 12 genes from the renin-angiotensin-aldosterone system (RAS) or previously implicated in blood pressure regulation. Significant associations were seen between one polymorphism in the angiotensin converting enzyme (ACE) gene, two polymorphisms in the angiotensin II type 2 receptor (AGTR2) gene, and clinical parameters of mean sitting diastolic and systolic blood pressure decrease. These effects were not found with irbesartan and placebo treatment, rather they were specific to aliskiren treatment in this analysis.

The clinical trial. A multicenter, randomized, double-blind, parallel group clinical trial was designed to explore the efficacy and safety of aliskiren compared to placebo in a population of mild-to-moderate hypertensive patients. The treatment was for eight weeks, i.e., the clinical trial endpoint was at eight weeks for this particular trial. The primary objective of the trial was is to determine the blood pressure lowering effects of aliskiren 150 mg, aliskiren 300 mg, and aliskiren 600 mg compared to 150 mg irbesartan and placebo in patients with mild-to-moderate essential hypertension. The demographic characteristics were generally comparable across treatment groups. The majority of patients were Caucasian and younger than 65 years of age, with a mean age in the mid 50s. The overall distribution of males and females was even.

The primary efficacy variable analysed was MSDBP change (reduction in MSDBP from baseline). The secondary efficacy variables analysed were MSSBP change (reduction in MSSBP from baseline), responder ratio, plasma renin activity (PRA) reduction and active plasma renin (AREN) increase from baseline.

Regarding primary efficacy, pairwise comparisons showed that all doses of active treatment were statistically superior to placebo in reducing mean sitting diastolic blood pressure (MSDBP) at Endpoint and at Week 8 in the intend-to treat (ITT) population, and at Endpoint in the per protocol population. Similar MSDBP reductions were achieved with aliskiren 150 mg and irbesartan 150 mg. Aliskiren 300 and 600 mg produced the greatest reductions, but no greater reductions were observed with the 600 mg dose.

Regarding secondary efficacy, for mean sitting systolic blood pressure (MSSBP), all doses of active treatment were statistically superior to placebo at Endpoint. At Week 8, aliskiren 300 and 600 mg were statistically superior to placebo and irbesartan at Endpoint. Similar MSSBP reductions were achieved with aliskiren 150 mg and irbesartan 150 mg. Aliskiren 300 and 600 mg produced the greatest reductions, but no greater reductions were observed with the 600 mg dose.

For proportion of successful responders (defined as MSDBP <90 mm Hg or a ≧10 mm Hg reduction from baseline; or MSSBP <140 mm Hg or a ≧20 mm Hg reduction from baseline) showed that all active treatments were statistically superior to placebo at Endpoint. MSDBP results for the ITT population at Endpoint were 59-67% responders for the aliskiren groups, vs. 56% for irbesartan and 38% for placebo. MSSBP results for the ITT population at Endpoint were 57-68% responders for the aliskiren groups vs 59% for irbesartan and 36% for placebo. In the ITT population, dose-related mean increases in active renin were observed in the aliskiren groups (20.8, 29.9 and 93.6 mu/mL for aliskiren 150, 300 and 600 mg, respectively). Aliskiren 150 mg produced a greater mean increase in renin than did irbesartan 150 mg (11.2 mu/mL). This is consistent with the mechanism of action for renin inhibitors.

Patients participating in the clinical trial were asked to provide a separate consent for participation in pharmacogenetic analysis. Blood samples from all consenting patient were collected at the individual trial sites and then shipped to Covance (Indianapolis, Ind., USA). The genomic DNA of each patient was extracted from the blood by Covance using the PUREGENE™ DNA Isolation Kit (D-50K) (Gentra, Minneapolis, Minn., USA). Ultimately, 511 patients were genotyped, with roughly equal ratios of each treatment arm.

Pharmacogenetic analysis objectives and design. Retrospective pharmacogenetic analysis was conducted on all patients who had consented to provide samples for the pharmacogenetic investigation: The primary goal was to identify associations between genetic variation and variations in efficacy of treatment with aliskiren, assessed primarily by MSDBP and secondarily by MSSBP and responder ratio.

A candidate gene approach was used to select 48 polymorphisms in 12 genes from the renin angiotensin system (RAS) or previously associated with blood pressure regulation (TABLE 1). All available samples were genotyped for each SNP. Association studies were then performed as described below.

TABLE 1
Candidate Genes Selected For Pharmacogenetic Analysis

| Gene Symbol | Gene Name |
|---|---|
| RENBP | renin binding protein |
| REN | renin |
| ACE | angiotensin I converting enzyme |
| | (peptidyl-dipeptidase A) 1 |
| ACE2 | angiotensin I converting enzyme |
| | (peptidyl-dipeptidase A) 2 |
| AGT | angiotensinogen |
| AGTR1 | angiotensin II receptor, type 1 |
| AGTR2 | angiotensin II receptor, type 2 |
| ABCC2/MRP2 | ATP-binding cassette, sub-family C (CFTR/MRP), |
| | member 2 |
| AGTRAP | angiotensin II receptor-associated protein |
| CYP11B2 | cytochrome P450, family 11, subfamily B, polypeptide 2, |
| | aldosterone synthase |
| TGFB1 | transforming growth factor, beta 1 |
| NOS3/eNOS | nitric oxide synthase 3 (endothelial cell) |

Genotyping. Single nucleotide polymorphism (SNP) assays were designed using information from the public dbSNP database and the proprietary Celera/ABI database. The resulting probe sets for the genotyping assay were generated for ABI's Assays-by-Design® platform. Livak K J, Marmaro J, & Todd J A, Nature Genetics 9: 341-2 (1995). Genotyping was performed on 10 ng of genomic DNA according to the manufacturer's instructions.

The list of polymorphisms genotyped in this analysis, including internal CPG locus code, gene, pathway, database reference and effect, is given in TABLE 2.

TABLE 2A
Genotyped Polymorphisms

| pg_locus_id | Gene | rs_number | Description |
|---|---|---|---|
| 5284 | ABCC2 | hCV11305436 | intronic |
| 4786 | ABCC2 | rs2273697 | A > G I417V |
| 4783 | ABCC2 | rs2756104 | intronic |
| 4784 | ABCC2 | rs2756109 | intronic |
| 4785 | ABCC2 | rs3740066 | T > C I1324I |
| 4782 | ABCC2 | rs717620 | untranslated |
| 4797 | ABCC2 | rs8187710 | A > G |
| | | | Y1515C |
| 5286 | ACE | hCV1247681 | intronic |
| 4766 | ACE | rs4293 | intronic |
| 4769 | ACE | rs4317 | C > T P32S |
| 4767 | ACE | rs4329 | intronic |
| 4768 | ACE | rs4362 | C > T F1129F |
| 400 | ACE | rs4364 | A > C S712R |
| 1345 | ACE | | ins/del |
| 4746 | ACE2 | rs2285666 | intronic |
| 1669 | ACE2 | rs879922 | intronic |
| 4773 | AGT | rs1926723 | intronic |
| 1667 | AGT | rs4762 | T > C M207T |
| 2 | AGT | rs699 | C > T T268M |
| 4772 | AGT | rs943580 | genomic |
| 4796 | AGTR1 | rs2638362 | intronic |
| 4780 | AGTR1 | rs3772616 | intronic |
| 411 | AGTR1 | rs5182 | C > T L191L |

TABLE 2B
Genotyped Polymorphisms

| pg_locus_id | Gene | rs_number | Description |
|---|---|---|---|
| 5287 | AGTR2 | hCV1841569 | |
| 1445 | AGTR2 | rs1403543 | untranslated |
| 186 | AGTR2 | rs5193 | untranslated |
| 4795 | AGTR2-LD | rs4497127 | genomic |
| 5288 | AGTRAP | hCV516817 | intronic |
| 4787 | AGTRAP | rs4073574 | genomic |
| 4789 | CYP11B2 | rs4539 | G > A R173K |
| 575 | CYP11B2 | rs4544 | C > T T339I |
| 4788 | CYP11B2 | rs4545 | A > G S435G |
| 3541 | CYP11B2 | rs6431 | intronic |
| 4792 | NOS3 | rs1007311 | intronic |
| 482 | NOS3 | rs1799983 | G > T E298D |
| 4791 | NOS3 | rs1800779 | genomic |
| 4464 | NOS3 | rs1800780 | intronic |
| 4793 | NOS3 | rs891512 | intronic |
| 5292 | REN | rs11571092 | intronic |
| 1513 | REN | rs1464816 | intronic |
| 4771 | REN | rs3730103 | intronic |
| 4794 | REN | rs6704321 | C > G P403A |
| 4770 | RENBP | rs2269371 | G > A G274D |
| 2407 | RENBP | rs2269372 | intronic |
| 1676 | RENBP | rs762656 | genomic |
| 5285 | TGFB1 | hCV11707868 | intronic |
| 4790 | TGFB1 | rs2241717 | intronic |
| 4798 | TGFB1 | rs8105161 | intronic |

Statistical analysis. Genotype-phenotype association studies and related analyses were performed in SAS (Cary, N.C., USA) using Analyst®.

Association tests used categorical genotypes as the independent variable, with no assumption about dominance, and the various efficacy variables as dependent variables. Tests of continuous dependent variables used an ANCOVA analysis, and logistic regression was used for categorical dependent variables. Covariates in the genotype-phenotype association analysis were: treatment, trial region and baseline measurement.

ANCOVA analysis was repeated with the same model for each treatment group. In addition, the percent of responders was analyzed by means of a logistic regression model with treatment and region as factors and baseline as a covariate. Associations with p<0.05 in the whole dataset were deemed significant.

Angiotensin converting enzyme (ACE). Ang I is converted to the active octapeptide Ang II by angiotensin converting enzyme (ACE). SNP_{—}4769 was genotyped in the patients together with six other polymorphisms in this critical enzyme in the RAS. Significant association was seen between SNP_{—}4769 and the primary efficacy variable of MSDBP reduction from baseline (p=0.0058), and with the secondary variable of responder ratio (p=0.001). For the other secondary variable of MSSBP reduction from baseline, there was not significant association but only the same trend seen (p=0.0745).

When ANCOVA analysis was repeated for each treatment group, the association was only detected for the aliskiren treated groups, but not the ibesartan or the placebo groups. No significant association was found between any SNPs and the secondary parameters plasma renin activity (PRA) and active renin (AREN). The aliskiren specific effects are shown in TABLE 3.

TABLE 3
Effect of ACE and AGTR2 Variations on MSDBP, MSSBP and
Responder Ratio in the Aliskiren Treated Groups

| | SNP_4769 | | | SNP_1445 | | | SNP_4795 | | |
|---|---|---|---|---|---|---|---|---|---|
| | TT | CT | CC | AA | AG | GG | GG | GA | AA |
| n | 278 | 14 | 0 | 106 | 69 | 118 | 106 | 72 | 115 |
| MSDBP reduction | −11.1 | −4.7 | NA | −11.2 | −13.2 | −9.3 | −11.1 | −13.3 | −9.0 |
| (mmHg) | | | | | | | | | |
| p | 0.0058* | | | 0.0074* | | | 0.0022* | | |
| MSSBP reduction | −13.7 | −6.2 | NA | −13.9 | −15.7 | −11.8 | −13.6 | −16.3 | −11.5 |
| p | 0.0384* | | | 0.1360 | | | 0.0479* | | |
| Responder ratio | 69% | 14% | NA | 74% | 71% | 58% | 74% | 73% | 56% |

| | | | |
|---|---|---|---|
| p | 0.0024* | 0.1399 | 0.0382* |

SNP_{—}4769 is a coding SNP which changes the amino acid sequence from proline to serine at codon 32 in the ACE enzyme. The ACE I/D polymorphism (presence or absence of a 287 by Alu repeat sequence in intron 16) has been associated with the regulation of ACE level and activity and its ramifications in the RAS. Rigat B et al., J. Clin. Invest. 86(4): 1343-6 (1990).

Angiotensin II receptor, type 2 (AGTR2). AGTR2 encodes the angiotensin II receptor, type II. The type I angiotensin II receptor is the target of several antihypertensive drugs. Ang II signals through binding to the type I receptor to induce vasoconstriction and blood pressure increase. The type II angiotensin II receptor has been shown to inhibit ACE activity and attenuate the type I receptor mediated actions, thereby cause vasodilation. Hunley T E et al., Kidney Int. 57(2): 570-7 (2000); Ichiki T et al., Nature 377(6551): 748-50 (1995).

Significant association was seen between the AGTR2SNPs (SNP_{—}1445 and SNP_{—}4795) and the primary efficacy variable of MSDBP reduction from baseline (p=0.0078 and 0.0004 respectively). SNP_{—}4795 also demonstrated significant association with the secondary variable of MSSBP reduction from baseline (p=0.0245), but not with the other secondary parameter of responder ratio. SNP_{—}1445 meanwhile did not show significant association with the secondary parameters. No significant association was found between any SNPs and the secondary parameters plasma renin activity (PRA) and active renin (AREN).

When ANCOVA analysis was repeated for each treatment group, the association was only detected for the aliskiren treated groups, but not the ibesartan or the placebo groups. The effects of genotype in the aliskiren treated patients are shown above in TABLE 3.

Both these two SNPs are non-coding. SNP_{—}1445 is in the untranslated region of the AGTR2 gene, and SNP_{—}4795 is from the genomic region with linkage disequilibrium to the AGTR2 gene.

Discussion. In summary, one variant in the ACE gene and two SNPs in the AGTR2 gene showed significant association with the primary parameter MSDBP. The ACE variant and one of the AGTR2 variant also showed significant association with the secondary parameters MSSBP and responder ratio. The second AGTR2 variant did not show significant association, but only the same trend to the secondary parameters. This genotype effect was only seen in the aliskiren treated groups, but not in the ibesartan or the placebo groups. Therefore, the above described effect is a pharmacogenetic effect specific to aliskiren.

The ACE variant effect is particularly interesting, because the difference between the TT and CT genotypes appear to be quite dramatic (MSDBP reduction 11.1 vs 4.7 mm Hg, MSSBP reduction 13.7 vs 6.2 mm Hg, and responder ratio 69% vs 14%). No CC homozygous genotype was seen among the patients, which makes the detected allele frequency much lower than the SNP DB reported allele frequency of 0.1.

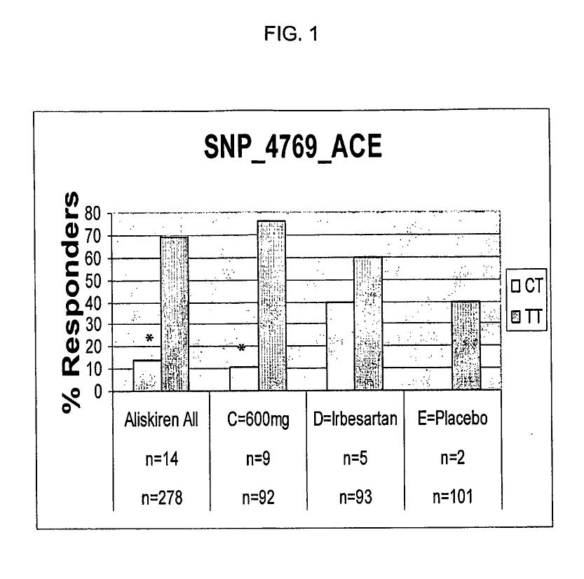

When segregating aliskiren response by genotype, the response ratio for the TT genotype increases to roughly 70%. The implication is that this result may help positioning aliskiren in the crowded antihypertensive market by supporting majority of the general population (about 80% according to SNP DB allele frequency, but may even be higher as seen in this analysis) having better response ratio, as shown in FIG. 1. It may also provide a way to help recruit potentially “better” responders to aliskiren in future studies.

The two SNPs in the AGTR2 gene are very likely in linkage disequilibrium.

These reported SNPs did not show association with the baseline measurements of MSDBP and MSSBP, again suggesting that the genetic effect is an aliskiren related pharmacogenetic effect.

### Example II

### Clinical Pharmacogenetics Analysis for Clinical Trial A2203

Introduction and summary. A retrospective pharmacogenetic analysis was conducted in an attempt to evaluate potential association between genetic variation and clinical outcome in a clinical trial. See, EXAMPLE I. Subsequently, the results of another clinical trial (A2203) was considered for replication analysis. Specifically, we examined 48 polymorphisms in 12 genes from the renin-angiotensin-aldosterone system (RAS) or previously implicated in blood pressure regulation.

In EXAMPLE I, significant associations were seen between one polymorphism in the angiotensin converting enzyme (ACE) gene, two polymorphisms in the angiotensin II type 2 receptor (AGTR2) gene, and clinical parameters of mean sitting diastolic and systolic blood pressure decrease. These effects were not found with irbesartan and placebo treatment. Additionally, for the ACE missense variant (Pro32Ser) associated with reduced response, we found a much higher C (Pro) allele frequency in blacks than Caucasians (19/154 vs. 2/790).

In this EXAMPLE, all four patients with the serine residue (C allele) were blacks. Due to the extremely small number of patients with C allele, An analysis could not be conducted for this SNP with the same model as in EXAMPLE I. In addition, the association of the two SNPs in AGTR2 found in EXAMPLE I were not replicated.

The clinical trial in this EXAMPLE (A2203) was a randomized, double-blind, multicenter, multifactorial, placebo-controlled, parallel-group study to evaluate the efficacy and safety of combinations of aliskiren and valsartan compared with their component mono-therapies and the combination of valsartan and hydrochlorothiazide (HCTZ) in hypertensive patients. The primary objectives of this EXAMPLE were to assess the blood pressure lowering effects of the combination of aliskiren and valsartan (75/80 mg, 150/160 mg and 300/320 mg) compared with their component monotherapies administered for 6 weeks in patients with clinic mean sitting diastolic blood pressure ([MSDBP] ≧95 mmHg and <110 mmHg) and to assess the blood pressure lowering effects of aliskiren 75 mg, 150 mg and 300 mg given alone versus placebo administered for 6 weeks in patients with clinic mean sitting diastolic blood pressure ([MSDBP] ≧95 mmHg and <110 mmHg). The treatment groups were generally comparable in demographics and baseline characteristics, with mean age 56 years, 56% male and 6.8% blacks.

The primary efficacy variable, change from baseline in MSDBP for aliskiren monotherapy vs. placebo, was statistically significant for the aliskiren 300 mg group. The overall magnitude of blood pressure lowering for both mono-therapies was consistent with that seen in previous studies. The magnitude of the placebo effect was however, higher than expected and higher than that seen in the previous aliskiren studies. Blood pressure lowering with the combination of aliskiren and valsartan was less than that observed with hydrochlorothiazide (HCTZ) 12.5 mg/valsartan, and the additivity was smaller at the maximum dose of aliskiren 300 mg/valsartan 320 mg than at lower strength combinations.

Pharmacogenetic analysis objectives. Retrospective pharmacogenetic analysis was conducted on all patients who had consented to provide samples for the pharmacogenetic investigation. The primary goal was to identify associations between genetic variation and variations in efficacy of treatment with aliskiren, assessed primarily by MSDBP and secondarily by MSSBP and responder ratio.

A candidate gene approach was used to select 48 polymorphisms in 12 genes from the RAS or previously associated with blood pressure regulation (TABLE 4). All available samples were genotyped for each SNP. Association studies were then performed as described in EXAMPLE I.

TABLE 4
Candidate genes selected for pharmacogenetic analysis

| Gene Symbol | Gene Name |
|---|---|
| RENBP | renin binding protein |
| REN | renin |
| ACE | angiotensin I converting enzyme |
| | (peptidyl-dipeptidase A) 1 |
| ACE2 | angiotensin I converting enzyme |
| | (peptidyl-dipeptidase A) 2 |
| AGT | angiotensinogen |
| AGTR1 | angiotensin II receptor, type 1 |
| AGTR2 | angiotensin II receptor, type 2 |
| ABCC2/MRP2 | ATP-binding cassette, sub-family C (CFTR/MRP), |
| | member 2 |
| AGTRAP | angiotensin II receptor-associated protein |
| CYP11B2 | cytochrome P450, family 11, subfamily B, polypeptide 2, |
| | aldostrerone synthase |
| TGFB1 | transforming growth factor, beta 1 |
| NOS3/eNOS | nitric oxide synthase 3 (endothelial cell) |

Samples. Patients participating in the clinical trial A2201 (see, EXAMPLE I) and clinical trial A2203 were asked to provide a separate consent for participation in pharmacogenetic analysis. Blood samples from all consenting patients were collected at the individual trial sites and then shipped to Covance (Indianapolis, Ind.). The genomic DNA of each patient was extracted from the blood by Covance using the PUREGENE™ DNA Isolation Kit (D-50K) (Gentra, Minneapolis, Minn.) and shipped to NIBR for genotyping. Ultimately, 511 patients from the clinical trial in EXAMPLE I (A2201) were genotyped, with roughly equal ratios of each treatment arm. Six hundred and eighty-four patients from A2203 were genotyped, with 3:1 ratio of patient numbers in placebo and aliskiren mono-therapy arms to other treatment arms.

The primary efficacy variable was MSDBP change (reduction in MSDBP from baseline at end point). The secondary efficacy variables are MSSBP change (reduction in MSSBP from baseline at end point), responder ratio, plasma renin activity (PRA) reduction and active plasma renin (AREN) increase from baseline.

Genotyping. SNP assays were designed using information from the public dbSNP database and the proprietary Celera/ABI database. The resulting probe sets for the genotyping assay were generated for ABI's Assays-by-Design® platform. Livak K J et al., Nat. Genet. 9: 341-2 (1995). Genotyping was performed on 10 ng of genomic DNA according to the manufacturer's instructions.

Genotyped polymorphisms. The list of polymorphisms genotyped in this study, including locus code, gene, database reference and effect, is given in TABLE 5.

TABLE 5
Genotyped polymorphisms

| pg_locus_id | gene | rs_number | Description |
|---|---|---|---|
| 5284 | ABCC2 | hCV11305436 | intronic |
| 4786 | ABCC2 | rs2273697 | A > G I417V |
| 4783 | ABCC2 | rs2756104 | intronic |
| 4784 | ABCC2 | rs2756109 | intronic |
| 4785 | ABCC2 | rs3740066 | T > C I1324I |
| 4782 | ABCC2 | rs717620 | untranslated |
| 4797 | ABCC2 | rs8187710 | A > G Y1515C |
| 5286 | ACE | hCV1247681 | intronic |
| 4766 | ACE | rs4293 | intronic |
| 4769 | ACE | rs4317 | C > T P32S |
| 4767 | ACE | rs4329 | intronic |
| 4768 | ACE | rs4362 | C > T F1129F |
| 400 | ACE | rs4364 | A > C S712R |
| 1345 | ACE | | ins/del |
| 4746 | ACE2 | rs2285666 | intronic |
| 1669 | ACE2 | rs879922 | intronic |
| 4773 | AGT | rs1926723 | intronic |
| 1667 | AGT | rs4762 | T > C M207T |
| 2 | AGT | rs699 | C > T T268M |
| 4772 | AGT | rs943580 | genomic |
| 4796 | AGTR1 | rs2638362 | intronic |
| 4780 | AGTR1 | rs3772616 | intronic |
| 411 | AGTR1 | rs5182 | C > T L191L |
| 5287 | AGTR2 | hCV1841569 | |
| 1445 | AGTR2 | rs1403543 | untranslated |
| 186 | AGTR2 | rs5193 | untranslated |
| 4795 | AGTR2-LD | rs4497127 | genomic |
| 5288 | AGTRAP | hCV516817 | intronic |
| 4787 | AGTRAP | rs4073574 | genomic |
| 4789 | CYP11B2 | rs4539 | G > A R173K |
| 575 | CYP11B2 | rs4544 | C > T T339I |
| 4788 | CYP11B2 | rs4545 | A > G S435G |
| 3541 | CYP11B2 | rs6431 | intronic |
| 4792 | NOS3 | rs1007311 | intronic |
| 482 | NOS3 | rs1799983 | G > T E298D |
| 4791 | NOS3 | rs1800779 | genomic |
| 4464 | NOS3 | rs1800780 | intronic |
| 4793 | NOS3 | rs891512 | intronic |
| 5292 | REN | rs11571092 | intronic |
| 1513 | REN | rs1464816 | intronic |
| 4771 | REN | rs3730103 | intronic |
| 4794 | REN | rs6704321 | C > G P403A |
| 4770 | RENBP | rs2269371 | G > A G274D |
| 2407 | RENBP | rs2269372 | intronic |
| 1676 | RENBP | rs762656 | genomic |
| 5285 | TGFB1 | hCV11707868 | intronic |
| 4790 | TGFB1 | rs2241717 | intronic |
| 4798 | TGFB1 | rs8105161 | intronic |

Statistical analysis. Genotype-phenotype association studies and related analyses were performed in SAS (Cary, N.C.) using Analyst.

Association tests used categorical genotypes as the independent variable, with no assumption about dominance, and the various efficacy variables as dependent variables. Tests of continuous dependent variables used an ANCOVA analysis, and logistic regression was used for categorical dependent variables. Note that none of the results have been adjusted for multiple hypothesis testing. A threshold of p<0.05 was used to define suggestive associations. Covariates in the genotype-phenotype association analysis were: (1) dose level of treatment; (2) trial region coded as STU1A in A2201 (see EXAMPLE I) or REGION in A2203; (3) baseline measurement of MSDBP and MSSBP; and (4) race. Analysis was performed in all the samples from the three aliskiren treated arms first. When suggestive associations were seen, the same analysis was done in the irbesartan and placebo arms.

TABLE 6
Demographic and baseline MSDBP characteristics of genotyped patients (mean ± SE)

| A2201 (see EXAMPLE I) | | | | | A2203 (this EXAMPLE) | | | |
|---|---|---|---|---|---|---|---|---|
| | | | | MSDBP | | | | MSDBP |
| Treatment | N = | BMI | Age | Baseline | Treatment | N = | Age | Baseline |
| Aliskiren | 99 | 31.01 ± 0.67 | 54.15C | 98.76 ± 0.34 | Aliskiren 75 mg | 102 | 54.73 ± 1.37 | 98.69 ± 0.28 |
| 150 mg | | | 1.30 | | Aliskiren 150 mg | 115 | 55.83 ± 1.18 | 99.47 ± 0.35 |
| Aliskiren | 96 | 30.79 ± 0.58 | 55.93 ± 1.03 | 99.16 ± 0.36 | Aliskiren 300 mg | 102 | 57.09 ± 1.22 | 99.26 ± 0.34 |
| 300 mg | | | | | Valsartan 80 mg | 39 | 58.08 ± 1.77 | 99.30 ± 0.61 |
| Aliskiren | 101 | 30.53 ± 0.65 | 55.97 ± 1.08 | 98.90 ± 0.38 | Valsartan 160 mg | 33 | 53.45 ± 2.15 | 98.87 ± 0.69 |
| 600 mg | | | | | Valsartan | 34 | 55.50 ± 1.65 | 99.16 ± 0.57 |
| Irbesartan | 99 | 31.81 ± 0.72 | 56.66 ± 1.21 | 99.41 ± 0.40 | 320 mg | | | |
| 150 mg | | | | | Aliskiren 75 mg and | 35 | 55.43 ± 1.95 | 99.73 ± 0.64 |
| Placebo | 103 | 30.65 ± 0.60 | 58.21 ± 1.15 | 98.89 ± 0.33 | Valsartan 80 mg | | | |
| | | | | | Aliskiren 150 mg and | 34 | 56.26 ± 1.94 | 99.32 ± 0.61 |
| | | | | | Valsartan 160 mg | | | |
| | | | | | Aliskiren 300 mg and | 41 | 57.10 ± 1.86 | 98.82 ±0.46 |
| | | | | | Valsartan 320 mg | | | |
| | | | | | Valsartan 160 mg and | 41 | 55.41 ± 1.93 | 98.83 ± 0.55 |
| | | | | | HCTZ 12.5 mg | | | |
| | | | | | Placebo | 108 | 55.80 ± 1.24 | 98.86 ± 0.29 |

ACE. SNP 4769 was genotyped in the patients together with six other polymorphisms in this critical enzyme in the RAS. Significant association was seen between SNP 4769 and the primary efficacy variable, MSDBP reduction from baseline (p=0.023), and with the secondary variable, responder rate (p=0.0048). For the other secondary variable, MSSBP reduction from baseline, there was no significant association but only the same trend seen (p=0.14).

When ANCOVA analysis was repeated with the same model for each treatment group, the association was only detected for the aliskiren treated groups, but not the ibesartan or the placebo groups. The aliskiren specific effects are shown in TABLE 7. No significant association was found between any SNPs and the secondary parameters plasma renin activity (PRA) and active rennin (AREN).

TABLE 7
Effect of ACE and AGTR2 variations on MSDBP, MSSBP and responder ratio in all aliskiren
treated groups in A2201 (* denotes significance, p < 0.05)

| | SNP_4769 | | | SNP_1445 | | | SNP_4795 | | |
|---|---|---|---|---|---|---|---|---|---|
| | TT | CT | CC | AA | AG | GG | GG | GA | AA |
| n | 278 | 14 | 0 | 106 | 69 | 118 | 106 | 72 | 115 |
| LSMEAN of MSDBP | −11.4 | −5.6 | NA | −11.0 | −13.3 | −9.4 | −10.8 | −13.4 | −9.2 |
| reduction (mmHg) | | | | | | | | | |
| p | | 0.023* | | | 0.0071* | | | 0.0026* | |
| LSMEAN of MSSBP | −11.7 | −5.9 | NA | −11.2 | −13.8 | −9.8 | −10.8 | −14.2 | −9.5 |
| reduction (mmHg) | | | | | | | | | |
| p | | 0.14 | | | 0.13 | | | 0.046* | |
| Responder rate | 70% | 14% | NA | 75% | 71% | 58% | 75% | 74% | 57% |

| | | | |
|---|---|---|---|
| p | 0.0048* | 0.17 | 0.060 |

SNP_{—}4769 is a coding SNP which changes the amino acid sequence from proline to serine at codon 32 in the ACE enzyme isoform 2 and 3. The ACE I/D polymorphism (presence or absence of a 287 by Alu repeat sequence in intron 16) has been associated with the regulation of ACE level and activity and its ramifications in the RAS. Rigat B et al., J. Clin. Invest. 86(4):1343-6 (1990). We tested ACE I/D in this exercise and observed no association to aliskiren response. SNP_{—}4769 is located in the 12^{th} intron of the ACE gene coding for the somatic isoform, and it is located in the first exon of the gene coding for the second and third isoforms. ACE I/D is located in the 16^{th} intron, which is in the same linkage disequilibrium block as SNP_{—}4769 in Caucasian, African American, and Chinese populations characterized (SNPbrowser, Applied Biosystems, Foster City, Calif.).

AGTR2. The type II angiotensin II receptor has been shown to inhibit ACE activity and attenuate the type I receptor mediated actions, thereby causing vasodilation. Ichiki T et al., Nature 377(6551):748-50 (1995); Hunley T E et al., Kidney Int. 57(2):570-7 (2000).

Significant association was seen between SNPs 1445 and 4795 and the primary efficacy variable, MSDBP reduction from baseline (p=0.0071 and 0.0026 respectively). SNP 4795 also demonstrated significant association with the secondary variable, MSSBP reduction from baseline (p=0.046), but not with the other secondary parameter, responder rate. SNP 1445 meanwhile did not show significant association with the secondary parameters. No significant association was found between any SNPs and the secondary parameters PRA and AREN.

When ANCOVA analysis was repeated with the same model for each treatment group, the association was only detected for the aliskiren treated groups, but not the ibesartan or the placebo groups. The effects of genotype in the aliskiren treated patients are shown above in TABLE 7.

When attempting to replicate the associations seen with these two SNPs in A2203 in the aliskiren treated arms, the results did not replicate (p>0.05). No trend of association was observed either. In the preliminary clinical study results summary, due to a higher than previously seen placebo response (8.6 mmHg), dose-response and magnitude of placebo-subtracted effects for aliskiren treatment was lower than expected. This inconsistency could have contributed, at least partially, to the lack of replication of the AGTR2 association.

Both these two SNPs are non-coding. SNP 1445 is in the untranslated region of the AGTR2 gene, and SNP_{—}4795 is from the genomic region with linkage disequilibrium to the AGTR2 gene. At least in Chinese, these two SNPs are in the same linkage disequilibrium block (SNPbrowser, Applied Biosystems, Foster City, Calif.).

Discussion. This analysis identified polymorphisms in two genes with significant associations with clinical outcome variables in the A2201 study (see, EXAMPLE I). One variant in the ACE gene and two SNPs in the AGTR2 gene showed significant association with the primary parameter MSDBP. The ACE variant and one of the AGTR2 variants also showed significant association with the secondary parameters MSSBP and responder ratio. The second AGTR2 variant did not show significant association but only the same trend to the secondary parameters. This genotype effect was only seen in the aliskiren treated groups, but not in the ibesartan or the placebo groups. Therefore, the above described effect suggests to be a pharmacogenetic effect specific to aliskiren.

The ACE Pro32Ser variant effect is particularly interesting, as the difference between the TT and CT genotypes appear to be quite dramatic (MSDBP reduction 11.4 vs 5.6 mm Hg, MSSBP reduction 11.7 vs 5.9 mm Hg, and responder ratio 70% vs 14%). However, no CC homozygous genotype was seen among all the patients. This makes the detected allele frequency much lower than the SNP DB reported allele frequency of 0.1, and the overall number of patients with the CT genotype on the very small side. We found a much higher C allele frequency in blacks than Caucasians (19/154 vs. 2/790). In A2203, all 4 patients with the serine residue (C allele) were blacks. Due to the extremely small number of patients with C allele, A2203 analysis could not be conducted with the same model.

ACE polymorphisms and their influence on ACE enzyme plasma concentration and blood pressure have been observed mostly in Africans and African-American populations. Bouzekri N et al., Eur. J. Hum. Genet. 12(6):460-8 (2004); Cox R et al., Hum. Mol. Genet. 11(23):2969-77 (2002); Zhu X et al., Am. J. Hum. Genet. 68(5):1139-48 (2001). The observation of higher allele frequency of SNP_{—}4769 in the ACE gene in black patients from the two SPP100 studies warrants a further investigation of the functional influence this polymorphism may have on ACE level or activity. Moreover, the amino acid change at codon 32 is speculated to be in the testis isoform of the enzyme.

When segregating aliskiren response by genotype, the response ratio for the TT genotype increases to roughly 70%. The implication is that this result may help position the drug in the crowded antihypertensive market by targeting majority of the general population (about 80% according to SNP DB allele frequency, may even be higher as seen in this study) who have the TT genotype associated with better response ratio, as shown in FIG. 2. It may also provide a way to help recruit potentially “better” responders to aliskiren in future studies.

The two SNPs in the AGTR2 gene are very likely in linkage disequilibrium, as the chromosome region around this locus is largely in one linkage disequilibrium block. The function of Ang II signalling via the type II receptor has been less studied compared to the type I receptor. This finding may further suggest AGTR2 involvement in Ang II function in blood pressure regulation downstream to renin inhibition.

These reported SNPs did not show association with the baseline measurements of MSDBP and MSSBP. This suggests that the genetic effect is an aliskiren related pharmacogenetic effect.

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

What is claimed is:

1. The use of aliskiren in the manufacture of a medicament for the treatment of hypertension in a selected patient population, wherein the patient population is selected on the basis of genetic polymorphisms in biomarker genes present in the patient, wherein the genetic polymorphisms are indicative of the efficacy of aliskiren in treating hypertension.

2. The use of claim 1, wherein the genetic polymorphisms are selected from the group consisting of SNP_{—}4769 in the angiotensin converting enzyme (ACE) gene; SNP_{—}1445 in angiotensin II receptor, type 2 (AGTR2) gene; SNP_{—}4795 in the AGTR2 gene; and combinations thereof.

3. A method for determining the responsiveness of an individual with hypertension to treatment with an antihypertensive agent, comprising the steps of;
(a) obtaining, for the two copies of genes present in the individual, the identity of nucleotide pairs at one or more polymorphic genetic loci, wherein the genetic loci are selected from the group consisting of the angiotensin converting enzyme (ACE) gene, the angiotensin II receptor, type 2 (AGTR2) gene, and combinations thereof; and
(b) assigning the individual to a “high” responder group if the nucleotide pairs at the polymorphic loci indicate that the individual is responsive to treatment with the antihypertensive agent.

4. The method of claim 3, wherein the genetic polymorphisms are selected from the group consisting of SNP_{—}4769 in the angiotensin converting enzyme (ACE) gene; SNP_{—}1445 in angiotensin II receptor, type 2 (AGTR2) gene; SNP_{—}4795 in the AGTR2 gene; and combinations thereof.

5. The method of claim 3, wherein the antihypertensive agent is aliskiren.

6. A method for treating hypertension in an individual, comprising the steps of:
(a) obtaining, for the two copies of genes present in the individual, the identity of a nucleotide pair at one or more polymorphic genetic loci, wherein the genetic loci are selected from the group consisting of the angiotensin converting enzyme (ACE) gene, the angiotensin II receptor, type 2 (AGTR2) gene, and combinations thereof; and
(b) either (i) administering an antihypertensive agent to the individual if the nucleotide pair at the polymorphic loci indicates that the individual is responsive to treatment with an antihypertensive agent, or (ii) administering an alternative therapy to the individual if the nucleotide pair at the polymorphic loci indicates that the individual is not responsive to treatment with an antihypertensive agent.

7. The method of claim 6, wherein the genetic polymorphisms are selected from the group consisting of SNP_{—}4769 in the angiotensin converting enzyme (ACE) gene; SNP_{—}1445 in angiotensin II receptor, type 2 (AGTR2) gene; SNP_{—}4795 in the AGTR2 gene; and combinations thereof.

8. The method of claim 6, wherein the antihypertensive agent is aliskiren.

9. A method for reducing mean sitting systolic blood pressure (MSSBP) in an individual, comprising the steps of:
(a) obtaining, for the two copies of genes present in the individual, the identity of a nucleotide pair at SNP_{—}4795 of the angiotensin II receptor, type 2 (AGTR2) gene; and
(b) either (i) administering an antihypertensive agent to the individual if the nucleotide pair at SNP_{—}4795 indicates that the individual is responsive to treatment with an antihypertensive agent, or (ii) administering an alternative therapy to the individual if the nucleotide pair at SNP_{—}4795 indicates that the individual is not responsive to treatment with an antihypertensive agent.

10. The method of claim 9, wherein the antihypertensive agent is aliskiren.

11. A method for reducing diastolic blood pressure in an individual, comprising the steps of:
(a) obtaining, for the two copies of genes present in the individual having a diastolic blood pressure in need of reduction, the identity of nucleotide pairs at one or more polymorphic genetic loci, wherein the genetic loci are selected from the group consisting of the angiotensin converting enzyme (ACE) gene, the angiotensin II receptor, type 2 (AGTR2) gene, and combinations thereof; and
(b) either (i) administering an antihypertensive agent to the individual if the identity of the nucleotide pairs at the one or more polymorphic genetic loci indicates that the individual is responsive to treatment with an antihypertensive agent, or (ii) administering an alternative therapy to the individual if the identity of the nucleotide pairs at the one or more polymorphic genetic loci indicates that the individual is not responsive to treatment with an antihypertensive agent.

12. The method of claim 11, wherein the antihypertensive agent is aliskiren.

13. Use of a gene product of a gene selected from the group consisting of the angiotensin converting enzyme (ACE) gene and the angiotensin II receptor, type 2 (AGTR2) gene as a target for drug activity, wherein the use comprises the steps of:
(a) contacting the drug with a first gene product encoded by a polynucleotide having nucleotide pair at a polymorphic site in the region of a selected gene indicating high responsiveness to treatment with aliskiren for hypertension;
(b) identifying the activity of the drug on said first gene product;
(c) contacting the drug with a second gene product encoded by a polynucleotide having nucleotide pair at a polymorphic site in the region of the selected gene indicating low responsiveness to treatment with aliskiren for hypertension;
(d) identifying the activity of the drug on said second gene product;
(e) identifying the similarities and differences between the activity identified in step (b) and the activity identified in step (d).


SEQUENCE LISTING (3 sequences)

SEQ ID NO: 1 - DNA, 140 bp, Homo sapiens
  variation (1)...(140): The nucleotide sequence of SNP_4769, a single nucleotide polymorphism of the angiotensin converting enzyme (ACE) gene
aggacttcccagcctcctcttcctgctgctctgctacgggcaccctctgctggtccccagccaggaggcycccaacaggtgacagtcacccatgggacaagcagccaggcaacaaccagcagccagacaaccacccacca

SEQ ID NO: 2 - DNA, 800 bp, Homo sapiens
  variation (1)...(800): The nucleotide sequence of SNP_1445, a single nucleotide polymorphism of the angiotensin II type 2 receptor  (AGTR2) gene
tggaaacttcattttttttgtttgagatttatttgaatgagctgttatgattggagacagtgagaatttcagattaatgttttgcagacaaaaaaaaacctctctggaaagctggcaagggttcataagtcagccctagaattatgtaggttgaaggctcccagtggacagaccaaacatataagaaggaaaccagagatctggtgctattacgtcccagcgtctgagagaacgagtaagcacagaattcaaagcattctgcagcctgaattttgaaggtaagtatgaacaatttatatataatttacttggaaagtagaacatacattaaatgaaaatattttttatggatgaacttctgtttttcctgtgttttaacactgtattttgcaaaactccraattatttagctgctgtttctcttacaggagtgtgtttaggcactaagcaagctgatttatgataactgctttaaacttcaacaaccagtaagtcttcaagtggaatttattattgattcttttatgttaatttgttaggtcaaaagaaaaatctttagagcaaaataaaagttttgctctttattaggaggttctttagatattacacttttaattgggtagcttatttgcatgtattttgaaactatctaaagtaaatagtgtttcctttgtatgcttatctttagctaatgtgtttttttttttggttttaaaataatgcttctagtgaaaaaaatcacaaaaacctcaacactgtaacgtttgagagcaacggctattcagttcggttaaaccgaa

SEQ ID NO: 3 - DNA, 399 bp, Homo sapiens
  variation (1)...(399): The nucleotide sequence of SNP_4795, a single nucleotide polymorphism of the angiotensin II type 2 receptor  (AGTR2) gene
ccaacacaaaagcacagcagttgagaactgggaaagcatcgcactacaactgctactgccattaaccacattgtcctggatgcccaagagcttaagagcccacttacctacctggtacactgctactacaactgacatctgagaaagccacccaaaggaacaagaatttccctgtctggaaccaacagaattgtcactarttctgtaccagatcccaaggatacacatgcttagcttactattactaccactgaaacttgcaaaagaacccatcaagcattccattccccagcacaaattcatcagtttctatcaataacctcacaatgccacacagaggaatagacagatactactaaggctgtttatagccaatgaaatcatacacagtcttcacca